(19) United States
(12) Patent Application Publication
(10) Pub. No.: US 2024/0277712 A1
(43) Pub. Date: Aug. 22, 2024

(54) TREATMENT AND PREVENTION OF DEVELOPMENTAL DISORDERS AND MENTAL DISEASES

(71) Applicants: KYUSHU UNIVERSITY, NATIONAL UNIVERSITY CORPORATION, Fukuoka-shi (JP); UNIVERSITY OF CALIFORNIA,SAN DIEGO, Oakland, CA (US)

(72) Inventors: Kinichi NAKASHIMA, Fukuoka-shi (JP); Hideyuki NAKASHIMA, Fukuoka-shi (JP); Alysson Renato MUOTRI, La Jolla, CA (US)

(73) Assignees: KYUSHU UNIVERSITY, NATIONAL UNIVERSITY CORPORATION, Fukuoka-shi (JP); THE REGENTS OF THE UNIVERSITY OF CALIFORNIA, Oakland, CA (US)

(21) Appl. No.: 18/569,721

(22) PCT Filed: Jun. 13, 2020
(86) PCT No.: PCT/JP2022/023638

§ 371 Date: Dec. 13, 2023

(30) Foreign Application Priority Data
Jun. 14, 2021 (JP) 2021-098585

(51) Int. Cl. A61K 31/513

(52) U.S. Cl. CPC A61K 31/513

(57) ABSTRACT

The present invention aims at therapeutics to treat and/or prevent Rett syndrome (RTT), autism spectrum disorders or schizophrenia for which no curative therapy has yet been found.

The invention relates to pharmaceutical compositions for preventing or treating Rett syndrome (RTT), autism spectrum disorders, or schizophrenia, which comprises a reverse transcription inhibitor for retrotransposon L1.

### TECHNICAL FIELD

The present patent application claims priority and benefit under the Paris Convention based on Japanese patent application No. 2021-098585, filed on Jun. 14, 2021, which is incorporated herein by reference in its entirety as if set forth in the above application.

The present invention relates to a pharmaceutical composition for treating and/or preventing a developmental disorder or a mental disease, and particularly, to a pharmaceutical composition for treating and/or preventing developmental disorders such as Rett syndrome and autism spectrum disorders, or mental diseases such as schizophrenia, which comprises a reverse transcription inhibitor for retrotransposon L1.

It is believed that environmental change factors such as maternal immune activation (MIA), maternal stress, maternal separation stress (MSS), and poor nurturing behaviors during early life stage (fertilization to young adulthood) are associated with an increased risk of developing psychiatric and neurological disorders including schizophrenia, autism spectrum disorders, depression, and anxiety disorders in the middle and late stages of life course (later adulthood). Although the details of the pathogenic mechanism thereof are unknown, the involvement of epigenetic modification changes has been postulated because changes in the early life stage appear as abnormalities in the middle and late stages of the life course, and the responsibility of retrotransposon LINE1 (long interspersed nucleotide elements or L1) has been reported in relation to epigenetic modifications (Non-Patent Document 1).

The retrotransposon L1 promoter is normally methylated, and L1 transcription is repressed by the binding of the methylated DNA-binding transcriptional repressor MeCP2 (methyl-CpG binding protein 2). However, when the L1 promoter is demethylated due to changes in environmental factors or other factors, MeCP2 dissociates and its transcription is activated. The present inventors found that L1 cDNA translocation usually occurs only in the brain among organs other than germ line, and that the expression of retrotransposon L1 is higher during differentiation of Neural Stem Cells (NSCs) into neurons (Non-Patent Document 2). In addition, non-patent literature 2 shows that L1 transcription is further enhanced in MeCP2-deficient NSCs, and that when the L1 promoter in NSCs is demethylated by the treatment with a DNA demethylating agent, MeCP2 does not bind to the same.

MeCP2 is located on the X chromosome, and the mutation thereof causes Rett syndrome (RTT), which a progressive psychiatric and neurological disease (Non-Patent Document 3) Rett syndrome was first reported in 1966 by Dr. Andreas Rett, who is a Viennese pediatric neurologist (Non-Patent Document 4). Subsequently, many cases were reported in the 1980s, and Rett syndrome has been recognized worldwide as a neurodevelopmental disorder that mainly affects girls.

Patent document 1 and its family of patent documents 2 and 3 describe a method for reducing non-LTR retrotransposons (non-Long terminal repeat (LTR) retrotransposons) in nerve cells by treating the nerve cells with an inhibitor for transposon. Retrotransposons are broadly classified into LTR retrotransposons and non-LTR retrotransposons, the latter being also called LINE1 or L1. In these patent documents, the nerve cells in question are in a certain disease state caused by retrotransposon L1 in the nerve cells, Rett syndrome is mentioned as a disease with nerve cells in this disease state, and a reverse transcriptase inhibitor is mentioned as an inhibitor for translocation. However, those patent documents do not specifically and explicitly describe that reverse transcriptase inhibitors enable to modulate retrotransposon L1 and to treat Rett syndrome, which has not been demonstrated by any experimental data.

### CITATION LIST

### Patent Documents

Patent Document 1: WO2011-017404A2
Patent Document 2: US 20130315886A1
Patent Document 3: US 20140038896A1

### Non-Patent Documents

Non-Patent Document 1: Suarez N A, Macia A & Muotri A R. Dev Neurobiol 78, 433-455 (2018)
Non-Patent Document 2: Muotri A R, Marchetto M C N, Coufal N G, Oefner R, Yeo G, Nakashima K, Gage F H. Nature 468, 443-446 (2010).
Non-Patent Document 3: Amir, R. E., Van den Veyver, I. B., Wan, M., Tran, C. Q., Francke, U., & Zoghbi, H. Y. Nat. Genet., 23, 185-188 (1999).
Non-Patent Document 4: Rett, A. Wien. Med. Wochenschr., 116, 723-726 (1966).

### SUMMARY OF THE INVENTION

### Problems to be Solved by the Invention

When treating developmental disorders such as Rett syndrome (RTT) and autism spectrum disorders, the primary treatment modalities are “medical education” and “adjustment of the living environment”, but pharmacotherapy may be necessary depending on the disease, disorder and symptoms in the target patient. For example, anti-epileptic drugs are prescribed for epileptic seizures; sleep aids, bipolar disorder medications, ADHD medications, and antipsychotics are prescribed for sleep disturbances, inattention, hyperactivity, impulsivity, self-injury, agitation, and aggression; and anxiolytics and antidepressants are prescribed for anxiety, depression, tension, and other psychiatric symptoms. However, any one of these drug therapies is merely symptomatic, and drug therapies that can resolve the underlying problem are always needed.

When treating a mental disease such as schizophrenia, drug therapies are conducted in reference to changes in monoamines and glutamate receptors. The symptoms of schizophrenia may be divided into three major categories: positive symptoms, negative symptoms, and cognitive dysfunction, and treatments are taken according to each symptom. Positive symptoms are treated primarily with drugs based on the dopamine hypothesis with some efficacy, but not always, whereas negative symptoms or cognitive dysfunction are not treated with any drug sufficiently with therapeutic efficacy.

In view of these situations, the present invention aims at new pharmaceutical compositions for treating and/or preventing developmental disorders such as Rett syndrome (RTT) and autism spectrum disorders or mental diseases such as schizophrenia.

### Means to Solve Problems

The inventors of the present application assumed that, in the conventional technology (Patent Documents 1-3) teaching that the retrotranscribed L1 cDNA is inserted into the genome to cause any abnormality in the expression and function of various genes, the treatment would not be possible unless the inserted L1 cDNA is removed from the genome since the onset of disease has occurred once the L1 cDNA was inserted into the genome. It should be noted that the process of removing specific genes from the genome is complicated and expensive. Under the circumstances, the present inventors conducted intensive studies, and have found that developmental disorders such as Rett syndrome (RTT) and autism spectrum disorders and mental diseases such as schizophrenia could be not only prevented, but also treated on the basis of the theory that the abnormal cellular response induced by the presence of the L1 cDNA, which is itself deemed as a functional molecule, could be alleviated by inhibiting the generation of the L1 cDNA with a reverse transcription inhibitor for retrotransposon L1, thereby accomplishing the present invention.

Accordingly, the present invention encompasses the following aspects:

### <Pharmaceutical Composition>

[1]

A pharmaceutical composition for treating and/or preventing a developmental disorder or a mental disease, which comprises a reverse transcription inhibitor for retrotransposon L1, wherein the inhibitor excludes a non-nucleoside reverse transcriptase inhibitor, nevirapine.

[2]

The pharmaceutical composition according to [1], wherein the reverse transcription inhibitor for retrotransposon L1 is a reverse transcriptase inhibitor.

[3]

The pharmaceutical composition according to [1] or [2], wherein the reverse transcriptase inhibitor is a nucleoside analog reverse transcriptase inhibitor.

[4]

The pharmaceutical composition according to any of [1] to [3], wherein the nucleoside analogue reverse transcriptase inhibitor is lamivudine or stavudine.

[5]

The pharmaceutical composition according to any of [1] to [4], wherein the developmental disorder is Rett syndrome.

[6]

The pharmaceutical composition according to any of [1] to [4], wherein the developmental disorder is an autism spectrum disorder.

[7]

The pharmaceutical composition according to any of [1] to [4], wherein the mental disease is schizophrenia.

### Effect of Invention

Innovative pharmaceutical compositions for developmental disorders such as Rett syndrome (RTT) and autism spectrum disorders, or mental diseases such as schizophrenia directed to the inhibition of retrotransposon L1 reverse transcription can be provided. The present invention enables therapeutics to treat and/or prevent developmental disorders such as Rett syndrome (RTT) and autism spectrum disorders, or mental diseases such as schizophrenia, for which no curative therapy has yet been found.

### BRIEF DESCRIPTION OF DRAWINGS

FIG. 1 is a graph that illustrates the effects of the administrations of 3TC, d4T and vehicle in the drinking water on neurological symptoms in MeCP2 knockout (−KO) mice over time. Data are shown as mean±s.e.m. KO, n=15; KO-3TC, n=10; KO-d4T, n=10. n: number of knockout mice used in the analysis. **p<0.01, ***p<0.001, KO vs. KO-3TC. †p<0.05, ††p<0.01, †††p<0.001, KO vs. KO-d4T. One-way ANOVA and Tukey's Multiple Comparison Test.

FIG. 2 is a graph that illustrates the effects of the administrations of 3TC, d4T and vehicle in the drinking water on survival in MeCP2-KO mice. KO, n=20; KO-3TC, n=13; KO-d4T, n=13. n: number of knockout mice used in the analysis. *p<0.05, KO vs. KO-3TC. ††p<0.01, KO vs. KO-d4T. Log rank test.

FIG. 3A (top) illustrates images of the neurons (black dots are Cell body (cell body)) of CTRL: neurons from normal human iPS cells, MECP2-KO: neurons from patient-derived iPS cells, MECP2-KO treated with the reverse transcriptase inhibitors 3TC and d4T (RTi), and MECP2-KO treated with nevirapine (NVP). The black dots comprise a nucleus within, and are only one in each condition, from which the neuronal projections extend. As such, each image shows a representative form of a single neuron. The complexity analysis of neurons by Soll analysis is shown in FIG. 3A (bottom).

FIG. 3B is a graph that illustrates that the reverse transcriptase inhibitors rescue the abnormal release of pro-inflammatory cytokine IL6 from MECP2-KO astrocytes. Astrocyte functionality was confirmed by the addition of IL1P to the media 48 hours before the IL6 expression analysis. Values are indicated so that IL6 expression in control (CTRL) (astrocytes derived from normal human iPS cells) is expressed as 1.

FIG. 3C (left) illustrates representative immunostained images of the neurons of control (CTRL), MECP2-KO (KO), MECP2-KO treated with reverse transcriptase inhibitors (KO+RTi), and MECP2-KO treated with NVP (KO+NVP) (MAP2 (gray), synapsin 1 (SYN1) (solid arrows), postsynaptic density protein 95 (PSD95) (dashed arrow), and synapses where SYN1 and PSD95 co-localize (bold solid open arrow)). Co-localized synaptic spot analysis is shown in FIG. 3C (right). Co-localized synaptic puncta per 20 μm on neurons in each condition were quantified. Data are shown as mean±s.e.m. CTRL, n=22; MECP2-KO, n=21; MECP2-KO+reverse transcriptase inhibitor, n=23; MECP2-KO+NVP, n=15. n: number of neurons traced per condition. *p<0.05, One-way ANOVA and Tukey's multiple comparisons test.

FIG. 3D is a graph that illustrates the rescue of neuronal activity (Weighted Mean Firing Rate, Hz) by reverse transcriptase inhibitors measured using multi-electrode arrays (MEA) with human iPS cell-derived neurons.

FIG. 3E (top) is representative images of cortical organoids from the varying treatment conditions used to measure organoid diameter. Scatterplot shows the quantification of the cortical organoid diameter from each condition. The error bars represent s.e.m. Control (CTRL), n=60; MECP2-KO, n=60; MECP2-KO+reverse transcriptase inhibitor, n=55; MECP2-KO+NVP, n=60. n: number of cortical organoids measured. *p<0.05, ***p<0.001. One-way ANOVA and Tukey's multiple comparisons test. FIG. 3E (bottom) illustrates the results of schematic representation of the cortical diameter measurements. Chronic treatment of cells with reverse transcriptase inhibitors rescues the size distribution to that of the control group.

FIG. 4 is a graph that illustrates the effect of 3TC administration on sociality in the MIA mouse model. Ctrl, n=15; MIA, n=8; MIA-3TC, n=3. n: Number of mice used in the analysis. ***p<0.001. t-Test.

FIG. 5A (left) is a representative image of cell bodies of prefrontal cortical neurons from 8-week-old WT mice (wild-type), MeCP2-KO (KO) mice, and 3TC-treated MeCP2-KO (KO-3TC) mice, all of which were Golgi-stained. The results of the quantified cell body area in the neurons of each mouse are shown in FIG. 5A (right). WT, n=76; KO, n=85; KO-3TC, n=88. n: Number of neurons used in the analysis. ***p<0.001. One-way ANOVA and Tukey's Multiple Comparison Test.

FIG. 5B (left) is representative images of dendritic spines of prefrontal cortical neurons in Golgi-stained 8-week-old WT, KO and KO-3TC mice. The results of the quantified spine density of neurons in each mouse are shown in FIG. 5B (right). WT, n=14; KO, n=14; KO-3TC, n=14. n: Number of neurons used in the analysis. *p<0.05, ***p<0.001. One-way ANOVA and Tukey's Multiple Comparison Test.

FIG. 6A is representative immunostained images of HEK293s subjected to the control (None-CTRL), those wherein L1-EGFP vector was overexpressed (L1-EGFP-CTRL), those wherein L1-EGFP vector was overexpressed and 3TC was added (L1-EGFP-3TC), those wherein L1-EGFP vector was overexpressed and d4T was added (L1-EGFP-d4T), and those wherein L1-EGFP vector was overexpressed, and tenofovir (Tenofovir disoproxil fumarate (TDF)) was added (L1-EGFP-TDF) (GFP (gray)).

FIG. 6B is a graph that illustrates the percentage of EGFP-positive cells (EGFP-positive cells/Hoechest (strained nucleus) positive cells) in each experimental group in FIG. 6A. None-CTRL, n=3; L1-EGFP-CTRL, n=3; L1-EGFP-3TC, n=3; L1-EGFP-d4T, n=3; L1-EGFP-TDF, n=3. n: Number of experiments for each condition. ***p<0.001, One-way ANOVA and Tukey's multiple comparisons test.

### EMBODIMENTS FOR CARRYING OUT INVENTION

In one embodiment, the present invention provides a pharmaceutical composition for treating and/or preventing developmental disorders such as Rett syndrome (RTT) and autism spectrum disorders or mental diseases such as schizophrenia, preferably Rett syndrome, which comprises a reverse transcription inhibitor, preferably a reverse transcriptase inhibitor, particularly a nucleoside analog reverse transcriptase inhibitor for retrotransposon L1, preferably lamivudine or stavudine.

The present inventors have found that inhibition of the retrotransposon L1 cDNA generation in nervous system cells enables to not only prevent but also treat developmental disorders such as Rett syndrome and autism spectrum disorders or mental diseases such as schizophrenia. As described above, Patent Documents 1-3 illustrate a method for reducing non-LTR retrotransposons in neural cells by treating neural cells with a transposon inhibitor, but those documents do not demonstrate the event with any specific evidence in the form of experimental data. Specifically, those documents describe that the amount of retrotransposon L1 DNA is higher in the brains of patients with Rett syndrome than in the brains in healthy persons. However, this shows that a number of cDNA of retrotransposon L1 that has been incorporated, i.e., transferred into genomic DNA should be higher, and does not show that a number of cDNAs of L1 in the cytoplasm, or the amount of reverse transcription itself would be higher. This is evident from e.g. FIG. 2 showing the results in transgenic mice crafted to express EGFP (a fluorescent protein) only when L1 translocation occur, and showing that the high number of cells expressing EGFP in various regions of the brain in MeCP2KO mice indicates that many translocations are occurring, i.e., the number of L1 incorporated into the genome is high.

Furthermore, Patent Documents 1-3 and the reports so far teach that the retrotransposon L1 cDNA is inserted into the genome, which disrupts the gene function to cause neurological diseases such as RTT. Based on this mechanism, the development of the disease leads to the destroyed gene function, and therefore the disease can be prevented, but cannot be treated with the transposon inhibitors and other substances described in Patent Documents 1-3. On the other hand, the present inventors have deemed the L1 cDNA itself reverse-transcribed in the cytoplasm as a functional factor, and thought that the inhibitory effect of retrotransposon L1 cDNA generation could treat abnormal cellular responses induced by the presence of the L1 cDNA, namely developmental disorders such as Rett syndrome (RTT) and autism spectrum disorders and mental diseases such as schizophrenia. The inhibitory effect of retrotransposon L1 cDNA generation can be obtained by reverse transcription inhibitors for retrotransposon L1.

According to the present invention, “reverse transcription inhibitor for retrotransposon L1” may be any substance that inhibits the generation of a reverse transcription product, cDNA, for the retrotransposon L1 in the cytoplasm of neural cells, as described above, and includes, for example, substances that inhibit the transcription of retrotransposon L1, the generation, translocation or function of L1 cDNA, or substances that inhibit them simultaneously. Reverse transcriptase inhibitors for L1 include an anti-retroviral drug used to treat HIV infections or AIDS, and in some cases, reverse transcriptase inhibitors that also treat hepatitis B may be exemplified. In other words, reverse transcriptase inhibitors are those that inhibit the activity of a reverse transcriptase, which is a viral DNA polymerase necessary for HIV and other retroviruses to replicate.

According to the present invention, examples of reverse transcriptase inhibitors include nucleoside or nucleotide analog reverse transcriptase inhibitors and non-nucleoside reverse transcriptase inhibitors.

Nucleoside or nucleotide analog reverse transcriptase inhibitors are analogs of natural deoxynucleotide that are required to synthesize viral DNAs, and compete with natural deoxynucleotides for incorporation into the viral DNA strands during the growth. However, unlike natural deoxynucleotide substrates, reverse transcriptase inhibitors lack a 3′-hydroxyl group in the deoxyribose moiety. As a result, after incorporation of the reverse transcriptase inhibitor, the next deoxynucleotide cannot form the next 5′-3′ phosphodiester bond needed to extend the DNA strand. Thus, incorporation of a reverse transcriptase inhibitor stops viral DNA synthesis. Preferably, it is a reverse transcriptase inhibitor with an inhibitory activity for L1 reverse transcriptase.

Examples of nucleoside analog reverse transcriptase inhibitors include: Zidovudine (also called AZT, ZDV and azidothymidine), didanosine (also called ddI), zalcitabine (also called ddC and dideoxycytidine), stavudine (also called d4T: Dai et al., BMC Biochemistry 2011, 12:18), lamivudine (also called 3TC, approved for treatment of both HIV and hepatitis B: Dai et al, BMC Biochemistry 2011, 12:18), abacavir (also called ABC), emtricitabine (also called FTC), entecavir (also called ETV), and tenofovir (also called TDF). Preferable examples include a nucleoside reverse transcriptase inhibitor with an inhibitory activity for L1 reverse transcriptase. More preferable examples include stavudine and lamivudine. Combinations of nucleoside analog reverse transcriptase inhibitors can also be used in the present invention: epzicom (EPZ) (Lamivudine+Abacavir [3TC+ABC]) truvada (TVD) (tenofovir+emtricitabine [TDF+FTC]) combival (CBV) (zidovudine+lamivudine [AZT+3TC]) descovy (DVY) (tenofovir alafenamide+emtricitabine [TAF+FTC]).

Non-nucleoside reverse transcriptase inhibitors block a reverse transcriptase by binding directly to the enzyme. Non-nucleoside reverse transcriptase inhibitors are not incorporated into viral DNA unlike nucleoside analog reverse transcriptase inhibitors, but instead inhibit the function of the domain in the reverse transcriptase protein, which is necessary to carry out the process of DNA synthesis.

Examples of non-nucleoside reverse transcriptase inhibitors include: Efavirenz (also called EFV; Dai et al., BMC Biochemistry 2011, 12:18), delavirdine (also called DLV; Dai et al., BMC Biochemistry 2011, 12:18), etravirine (also called ETR), rilpivirine (also called RPV), dravirine (also known as MK-1439). Preferable examples include a non-nucleoside reverse transcriptase inhibitor with an inhibitory activity for L1 reverse transcriptase. More preferable examples include efavirenz and delavirdine, which have an inhibitory activity for L1 reverse transcriptase. Even more preferable examples include etravirine or rilpivirine. Nevirapine (NVP) is a non-nucleoside reverse transcriptase inhibitor and has an inhibitory activity for HIV reverse transcriptase, but is not used in any compositions for treating developmental disorders and mental diseases according to the present invention since it does not have any inhibitory activity for L1 reverse transcriptase (Dai et al., BMC Biochemistry 2011, 12:18).

The following combinations can also be used in the present invention:

$$complera ( CMP ) ( rilpivirine + tenofovir + emtricitabine \{ RPV + TDF + FTC ] ) .$$

The first-generation non-nucleoside reverse transcriptase inhibitors, NVP, EFV, and DLV, differ in the chemical structural formula of each compound, but are similar in their combined states, which are likened to a butterfly with its wings spread. They can be classed into the two types based on the coupling form:
Tight-binding inhibitor: This is an agent that does not come off once the agent binds to RT, and EFV falls under this type. Tight-binding inhibitor is being considered for use as a disinfectant for prevention of HIV infections.
Rapid equilibrium inhibitor: This is an agent reversibly bound to RT and exhibits anti-HIV-1 effects due to extreme equilibrium bias on the side of RT and drug binding, but does not act as a bactericide. NVP and DLV fall under this type.

The second-generation non-nucleoside reverse transcriptase inhibitors, ETR and RPV, are diarylpyridine (DAPY) analogs and have a structure with characteristic flexibilities such as wiggling by twisting and repositioning by jiggling. The reason for the lack of cross-resistance with the first-generation NNRTIs is thought to be that these flexibilities allow to bind to the deformed binding site after acquisition of the resistance mutation at multiple sites, thereby inhibiting reverse transcriptase activity.

According to the present invention, examples of reverse transcriptase inhibitors include, in addition to the above-mentioned substances, prodrugs with improved oral absorption, modified substances that are not easily degraded by nucleases or esterases and maintain stable blood levels in vivo for a long time, new compounds that are effective against resistant strains, and any combinations thereof, all of which are also included in the “reverse transcription inhibitors for retrotransposon L1” according to the present invention, as far as they inhibit the generation of reverse transcription product cDNA for retrotransposon L1 in the cytoplasm of cells in the nervous system.

The target disorders to be treated and/or prevented with the pharmaceutical compositions of the present invention are developmental disorders such as Rett syndrome and autism spectrum disorders, and mental diseases such as schizophrenia, preferably Rett syndrome. As described above, the inventors have confirmed the inhibitory effect of L1 cDNA generation and thereby believe that the inhibition of the L1 cDNA generation can treat the disease with deeming the L1 cDNA itself reverse-transcribed in the cytoplasm as a functional factor. In MeCP2-KO mice, a known mouse model of Rett syndrome (Chen, RZ, Akbarian S, Tudor M and Jaenisch R. Nat Genet 27, 327-331 (2001)), the reverse transcriptase inhibitors 3TC and d4T, which are clinically used for the treatment of HIV, were demonstrated to markedly relieve typical symptoms of Rett syndrome and significantly increase in survival in these treated mice, as described in detail in the working examples. MeCP2-KO mice exhibit Rett syndrome symptoms due to the increased transcription of L1 (Non-Patent Document 2), and the working examples show that the typical symptoms of Rett syndrome were relieved by inhibiting the reverse transcription of L1 with a reverse transcriptase inhibitor.

It has been reported that leukocytes derived from peripheral blood from patients with autism spectrum disorder (ASD) exhibit the reduced methylation of L1 and the increased transcription thereof (Tangsuwansri C, et al., PLoS One 13, e0201071 (2018), Shpyleva S, et al., Mol Neurobiol 55, 1740-1749 (2017)), and that ASD patients with a developmental disorder also show the increased L1 expression (Suarez et al., Dev Neurobiol 434-455 (2018)), indicating that L1 is associated with symptoms of ASD. In the brains of ASD patients, L1 translocation is also frequent, and probably the transcription is also higher. These documents suggest that the reverse transcription inhibitors for retrotransposon L1 according to the present invention can treat and/or prevent ASD, similar to Rett syndrome.

It has been reported that the translocation of L1 cDNA in the postmortem brains of schizophrenia patients is increased (Bundo M, et al., Neuron 81, 306-313 (2014)). It has been also reported that L1 transcription is enhanced in the offspring born from the mother mice, to which have been prepared by administering polyI:C as creating mouse models of ASD and schizophrenia (Bundo M, et al., Neuron 81, 306-313 (2014)). These documents suggest that the reverse transcription inhibitors for retrotransposon L1 according to the present invention can treat and/or prevent schizophrenia, similar to Rett syndrome.

A case of Rett syndrome (RTT) was first reported in 1966 by a Viennese pediatric neurologist, Andreas Rett (Non-Patent Document 4). Subsequently, many cases were reported in the 1980s, and Rett syndrome became recognized worldwide as a neurodevelopmental disorder that occurs primarily in girls. The symptoms and severity of the disease vary widely from patient to patient, and the typical patients, who account for about 80% or more of the patients with the disease, appear normal until about 6 months of age, after which they develop autistic symptoms such as poor response to the outside world and difficulty in eye contact, and other symptoms, including soft body and delayed motor skills such as crawling on all fours and walking. The disease is a designated incurable disease (156) with symptoms including convulsions, abnormal breathing, and slow growth of head circumference, which are not always present, but are frequent (https://www.mhlw.go.jp/file/05-Shingikai-10601000-D aijinkanboukouseikagakuka-Kouseikagakuka/0000084170.pdf).

Autism spectrum disorder is a complex developmental disorder characterized by impaired social and communication skills, repetitive behaviors, unbalanced interests, and the like. ASD is thought to be caused by a combination of different genetic and environmental factors, and the pathogenesis thereof is extremely complex. The latest research has reported that the frequency of ASD in the United States is one in 68 children under the age of 8. Despite this high incidence, the pathogenesis of ASD remains largely unclear.

Schizophrenia is a mental disorder characterized by loss of contact with reality, hallucinations, delusions, and the like. The disease course is divided into prodromal, acute, rest and recovery phases. Positive symptoms are prominent in the acute phase, and negative symptoms such as lethargy and doing nothing become noticeable, entering into the rest period. It has also been suggested that cognitive dysfunction worsens in multiple stages. The disease is thought to involve an imbalance of neurotransmitters in the brain, the cause of which is unknown, and the disease tends to respond better to treatment if the treatment starts early.

According to the present invention, “treatment” means a method or process aimed at (1) delaying the onset of developmental disorders such as Rett syndrome and autism spectrum disorders or mental diseases such as schizophrenia; (2) slowing down or halting the progression, aggravation or exacerbation of the symptoms of developmental disorders such as Rett syndrome and autism spectrum disorders or mental diseases such as schizophrenia; (3) inducing remission of the symptoms of developmental disorders such as Rett syndrome and autism spectrum disorders or mental diseases such as schizophrenia; or (4) facilitating the cure of developmental disorders such as Rett syndrome and autism spectrum disorders or mental diseases such as schizophrenia. Treatment may be given as a prophylactic measure before the onset of the disease or the condition, or treatment may be given after the onset of the disease.

According to the present invention, “prevention” means a prophylactic action on the onset of developmental disorders such as Rett syndrome and autism spectrum disorders or mental diseases such as schizophrenia.

The pharmaceutical compositions of the present invention may be formulated by methods well-known to those skilled in the art. The pharmaceutical compositions of the invention can be administered by both parenteral and oral administration. For parenteral administration, for example, the compositions may be in the form of an injection type, nasal administration type, pulmonary administration type, or transdermal administration type. For example, the compositions may be administered systemically or locally by intravenous injection, intramuscular injection, intraperitoneal injection, subcutaneous injection and the like. For oral administration, the compositions can be in the forms of tablets, capsules, rounds, granules, dispersions, syrups, and the like.

The administration method may be appropriately selected depending on the patient's age and symptoms. The dose and the administration method may vary depending on the patient's weight, age, symptoms and other factors, and those skilled in the art can determine an appropriate dose and an administration method taking account of those conditions.

Substances for treating and/or preventing developmental disorders such as Rett syndrome and autism spectrum disorders or mental diseases such as schizophrenia should be effectively prophylactic or therapeutic agents for developmental disorders such as Rett syndrome and autism spectrum disorders or mental diseases such as schizophrenia.

In another aspect, the present invention relates to a method for treating and/or preventing developmental disorders or mental diseases, which comprises administering a reverse transcription inhibitor for retrotransposon L1 to a subject in need of such treatment or the like, preferably a method wherein the reverse transcription inhibitor for retrotransposon L1 is a reverse transcriptase inhibitor, and more preferably, a method wherein the reverse transcriptase inhibitor is a nucleoside analogue reverse transcriptase inhibitor.

Further, in another aspect, the invention relates to a reverse transcription inhibitor for retrotransposon L1, preferably a reverse transcriptase inhibitor, more preferably a nucleoside analogue reverse transcriptase inhibitor, for the treatment and/or prevention of developmental disorders or mental diseases.

In yet another aspect, the invention relates to the use of a reverse transcription inhibitor for retrotransposon L1 to manufacture a medicament for treating and/or preventing developmental disorders or mental diseases, preferably the use wherein the reverse transcription inhibitor for retrotransposon L1 is a reverse transcriptase inhibitor, more preferably the use wherein the reverse transcriptase inhibitor is a nucleoside analogue reverse transcriptase inhibitor.

### EXAMPLES

Hereinafter, the present invention will be described in detail by way of the working examples, but it should be noted that these would not limit the scope of the present invention and merely illustrate the invention.

### Example 1

Effects of reverse transcription inhibitors for retrotransposon L1 on MeCP2-KO mouse phenotype

Treatment with reverse transcription inhibitors was performed as follows.

MeCP2-KO mice (C57BL/6 background) were obtained from Mutant Mouse Resource & Research Centers (MMRRC) (Chen, RZ, Akbarian S, Tudor M and Jaenisch R. Nat Genet 27, 327-331 (2001)). All aspects of animal care and treatment were carried out according to the guidelines of the Experimental Animal Care Committee of Kyushu University.

Mice were maintained in a 12-hour light/dark cycle with free access to food and water. MeCP2-KO mice were divided into three groups of animals, each of which was administered with 3TC (QA-4615, Combi-blocks), d4T (d3580, Tokyo Chemical Industry Co., Ltd.) and vehicle. The mice were orally administered with 3TC (2 mg/ml), d4T (2 mg/ml) and vehicle in drinking water, respectively, starting at week 4. As a control, the vehicle-treated MeCP2-KO mice were used. All mice were observed daily.

Symptom scoring was conducted as follows.

Neurological symptoms of the mice were scored as described in Guy J, Gan J, Selfridge J, Cobb S and Bird A. Science 315, 1143-1147 (2007). In brief, the mice bred as described above were scored blind to genotype and treatment status, focusing on the six symptoms of mobility, gait, hindlimb clasping, tremor, breathing, and general condition. Each of the six symptoms was scored from 0 to 2:0 corresponds to symptom's being absent or the same as in the wild-type, 1 corresponds to symptom's being present, and 2 corresponds to severe symptom.

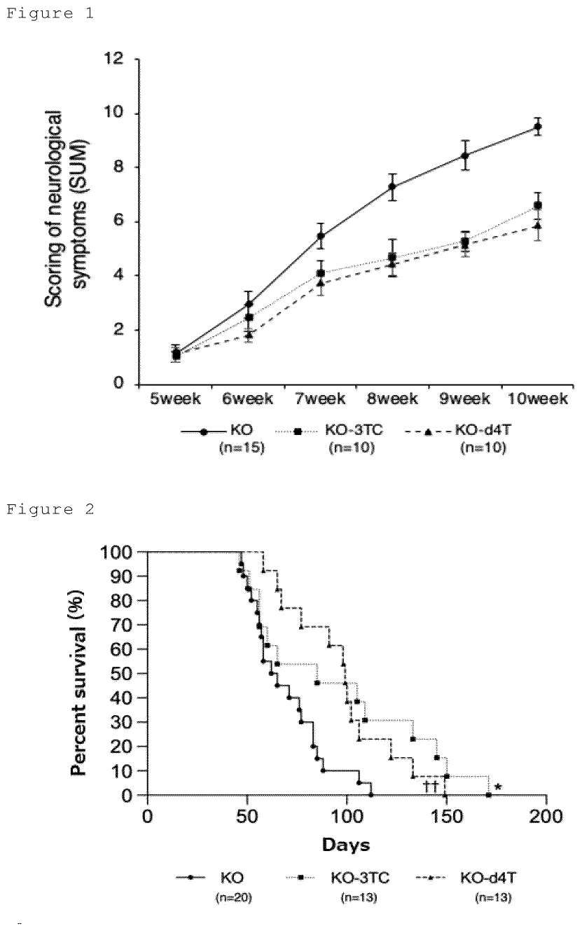

Six neurological symptoms scored in each of the three groups of the mice (vehicle, 15 mice; 3TC, 10 mice; d4T, 10 mice) were summed, and observed and recorded for up to 10 weeks. The results are shown in FIG. 1. Significant reductions of Rett symptoms were observed in the 3TC and d4T groups.

Next, the survival rate of the mice in each group (vehicle, 20 mice; 3TC, 13 mice; d4T, 13 mice) was examined. The result is shown in FIG. 2. Significant life extensions were observed in the 3TC and d4T groups.

### Example 2

### Effects of 3TC and d4T on iPS Cell-Derived Differentiated Nervous System Cells

Using human iPS cell-derived nervous system cells, it was found that the reverse transcriptase inhibitors 3TC and d4T were co-added to improve cell dysfunction.

Specifically, differentiated nervous system cells derived from normal human iPS cells and iPS cells obtained from patients suffered from Rett syndrome (hereinafter referred to as MECP2-deficient or MECP2-KO because the MECP2 gene was inactivated) were cultured and differentiated into cortical neurons. For differentiation into neurospheres (neural stem cell clusters), NPCs were dissociated from the plate using StemPro Accutase (Life Technologies), and 3-5 million cells were then cultured in suspension on 6-well plates in a shaker incubator at 95 r.p.m. at 37° C. for 48 hours in neural culture (NG) media (DMEM/F12, 1% Glutamax (Life Technologies), 1% N2 Neuroplex (Gemini Bio-products), 2% Gem21 NeuroPlex (Gemini Bio-products), 1% penicillin-streptomycin) supplemented with bFGF (basic Fibroblast Growth Factor, R&D Systems). After 48 hours, the media was replaced with NG media withdrawn with bFGF, and neurospheres were cultured for 2 weeks. iPS cell-derived cortical organoids were differentiated following (Trujillo et al., Cell Stem Cell 25, 558-569 (2019)). The cultured iPS cells were dissociated from the plate using a 1:1 Dulbecco's phosphate-buffered saline (DPBS, Fisher Scientific) and StemPro Accutase solution, transferred to 6-well plates, and kept in suspension. Neural induction media consisted of DMEM/F12, 1% Glutamax, 1% N2 Neuroplex, 1% non-essential amino acids (Gibco), 1% penicillin-streptomycin, 1 μM Dorsomorphin (R&D Systems), and 10 μM SB431542 (SB, Stemgent) Neural proliferation media consisted of Neurobasal media (Life Technologies), 2% Gem21 Neuroplex, 1% non-essential amino acids, 1% Glutamax, 20 ng/mL epidermal growth factor (EGF, Peprotech), and 20 ng/mL bFGF. For neuronal maturation, the cells were kept in the same media but the growth factors were withdrawn. Organoid results are combined from four separate batches of differentiation.

The differentiated nervous system cells were maintained for 8 weeks after FGF withdraw. Main significant features found for in vitro neurons derived from MECP2-KO cells are fewer synapses, smaller cell body size and changes in neuronal morphology (Marchetto et al., Cell 143, 527-539 (2010)).

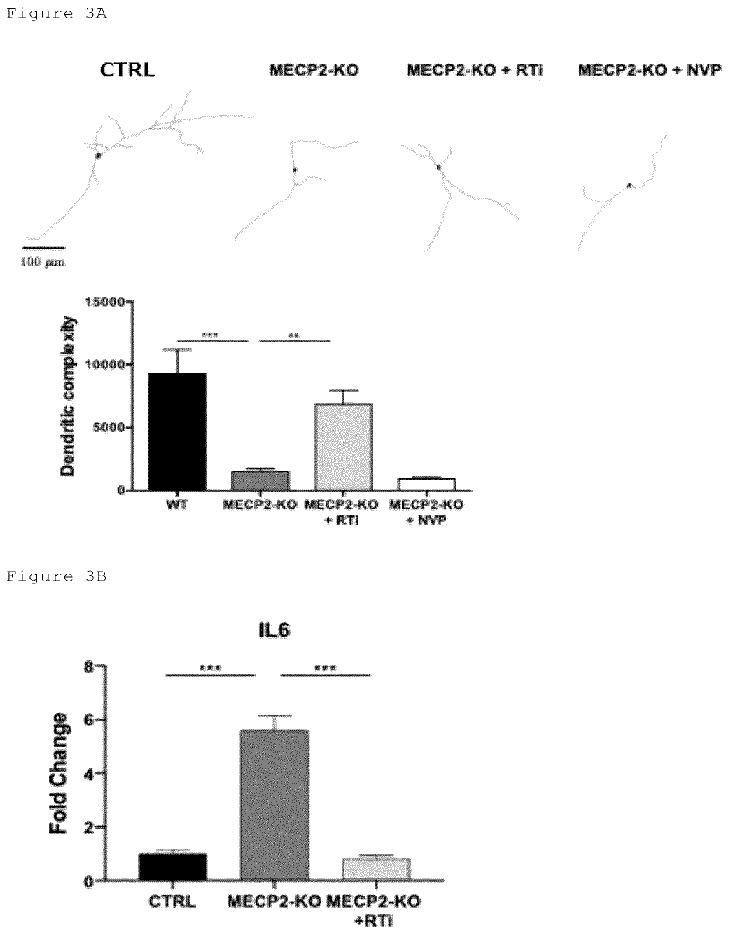

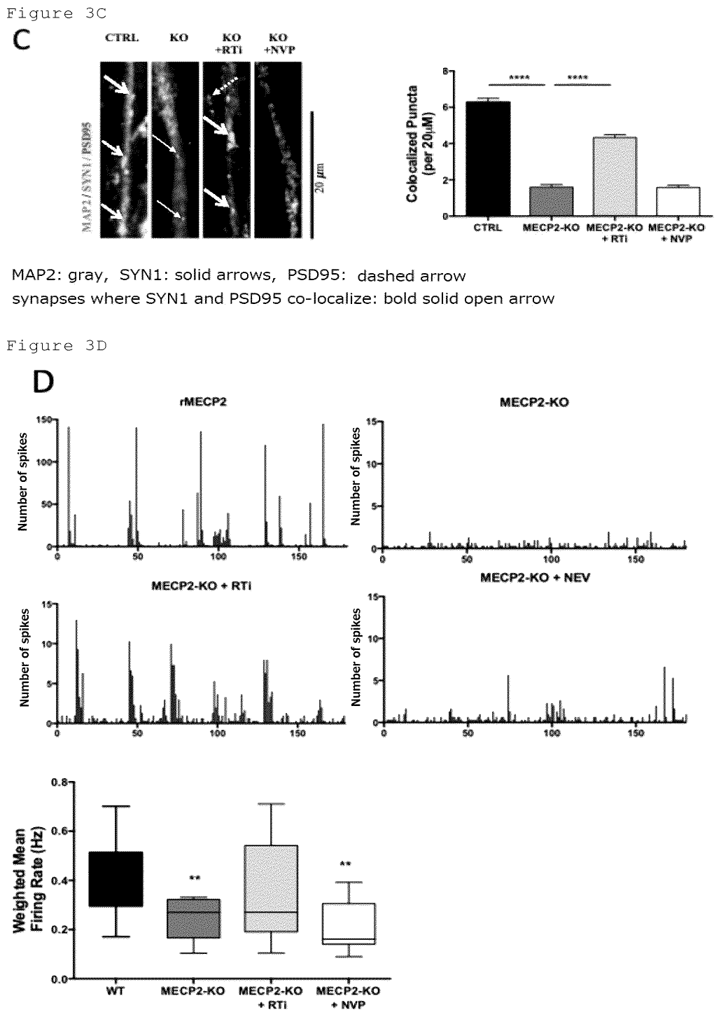

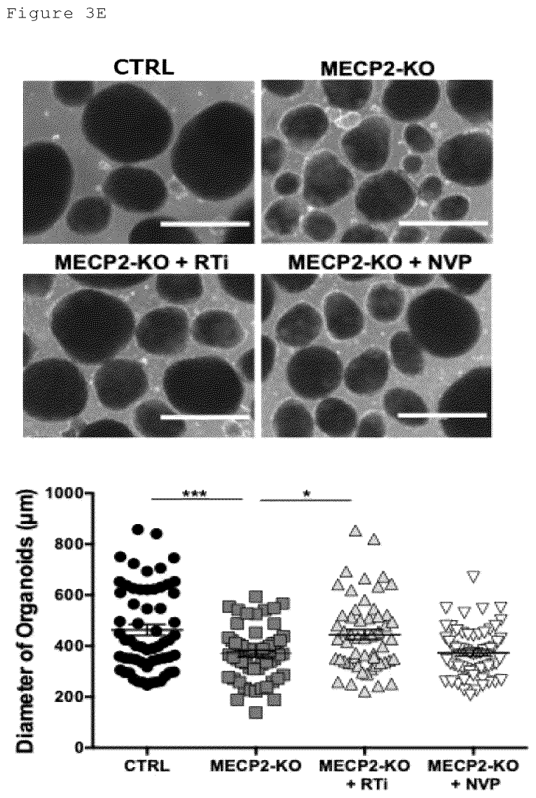

Next, 3TC and d4T were added to the obtained differentiated neural cell culture. The reverse-transcriptase inhibitors for cultured cells were prepared for as follows: Lamivudine (3TC, Sigma-Aldrich) was prepared in dimethyl sulfoxide (DMSO, Sigma Aldrich) and the preparation was added to media so that the final concentration of 3TC was adjusted into 10 μM. Stavudine (d4T, Sigma-Aldrich) was suspended in water and a final concentration of 1 μM d4T was used in combination with 3TC. Nevirapine (NVP) (Sigma Aldrich, SML0097), a non-nucleoside reverse transcriptase inhibitor that has an HIV reverse transcriptase inhibitory activity but was shown not to have any L1 reverse transcriptase inhibitory activity in Dai et al., BMC Biochemistry 2011, 12:18 SML0097, was suspended in DMSO and an final concentration of 400 nM nevirapine was used in media. Control cells (CTRL) contained 10 pjM DMSO as a vehicle control. The results obtained are shown in FIG. 3A.

Neurons from patient-derived iPS cells (MECP2-KO), i.e., MECP2-deficient neurons, displayed a decrease in dendritic length and number of branch points and a decrease in dendritic complexity when compared to neurons derived from normal human iPS cells (CTRL). In contrast, MECP2-deficient neurons treated with reverse transcriptase inhibitors displayed a statistically significant increase in cell body area and number of branch points, whereas this rescue was not seen in MECP2-deficient neurons treated with NVP. The result that the treatment with NVP did not rescue was as previously reported (Dai et al., BMC Biochem 12, 18 (2011); Jones et al., PLoS One 3, 31547 (2008)). In order to assess whether treatment with reverse transcriptase inhibitors affected control cells, they were treated chronically and subjected to morphological analysis. Results showed no difference when control cells were treated with reverse transcriptase inhibitors, suggesting that these antiretroviral drugs do not affect control neurons, but improve the RTT phenotype (FIG. 3A).

Soll analysis (Scholl analysis) was used to investigate differences in dendritic complexity under various conditions. Control neurons are significantly more complex when compared to MECP2-deficient neurons (MECP2-KO). Treating MECP2-deficient neurons (MECP2-KO) with reverse transcriptase inhibitors rescued dendrite length, number of branch points, cell body area and neuronal complexity. This indicates that reverse transcriptase inhibitors could ameliorate altered neuronal morphology and complexity in MECP2-deficient cells. Additionally, MECP2-deficient neurons treated with reverse transcriptase inhibitors displayed a significantly increase in co-localized puncta, which was indicative of synapse formation, when compared to neurons treated without reverse transcriptase inhibitors, which is not seen in NVP-treated MECP2-deficient neurons. No increase in co-localized puncta was observed in control cells treated with reverse transcriptase inhibitors (FIG. 3C).

Then, the present inventors examined whether reverse transcriptase inhibitors affect the ability to generate IL6 in MECP2-KO astrocytes that abnormally release a pro-inflammatory cytokine IL6. IL1P, which is known to induce IL6 generation from astrocytes was added to control cells derived from normal human iPS cells; CTRLs, MECP2-deficient neurons; MECP2-KO, and MECP2-deficient neurons treated with a reverse transcriptase inhibitor; MECP2-KO, and after 48 hours, IL6 expressions were determined and compared. The results are shown in FIG. 3B. MECP2-KO cells displayed that IL6 expression was abnormally upregulated nearly 6-fold compared to CTRLs (abnormal enhancement of IL6 generation capacity), whereas MECP2-KO cells treated with a reverse transcriptase inhibitor displayed that the expression was reduced to the same level as CRTLs (improved).

To fully understand if the changes observed in morphology and synaptogenesis lead to an improvement in neural connectivity, a more complex cellular model was utilized for analysis. NPCs were differentiated into 3-dimensional spheroidal cell masses (spheroids). The NPCs used to generate spheroids were chronically treated under their respective conditions and plated on multi-electrode array (MEA) planes to measure neural network activity. MEA plates were recorded weekly and analysis of spontaneous neural activity was performed after 4 weeks. Raster plots displayed time stamps of spikes across multiple channels, which showed that control spheroids exhibited higher number of spikes and spontaneous neural activity when compared to MECP2-deficient spheroids. Accordingly, weighted mean firing rate is significantly decreased only in MECP2-deficient spheroids and NVP-treated spheroids. Treatment with reverse transcriptase inhibitors in of MECP2-deficient spheroids improved the firing rate comparable to control levels. No differences were observed when control spheroids were treated with reverse transcriptase inhibitors. Taken together, the data suggests that the decrease in spontaneous neural activity seen in MECP2-deficient neurons is rescued with the antiretroviral treatment (FIG. 3D).

RTT is often associated with a microcephalic phenotype, in which the brain does not develop properly resulting in a smaller than a normal head. Therefore, the present inventors investigated whether a 3-dimentional model could mimic the microcephalic phenotype observed in patients. 3D cortical organoids, differentiated from human iPS cells displays a spontaneous self-assembled structure similar to a human neocortex and a transcriptional profile similar to a mid-fetal prenatal human brain. After 56 days of the differentiation process, when the organoids mature, the MECP2-deficient organoids exhibited a statistically significant reduction in diameter when compared to control organoids, but the above reverse transcriptase inhibitor treatment rescued the cortical organoid size, which was not seen with NVP. No significant changes were observed when control cells were treated with reverse transcriptase inhibitors (FIG. 3E).

Morphological analysis of iPS cell-derived neurons was performed as follows.

Cultured NPCs were transduced with a lentivirus backbone containing Synapsin 1 (SYN) promoter upstream of an EGFP reporter (Nageshappa et al., Mol Psychiatry 21, 178-188 (2016)). The transduction multiplicity of infection (M.O.I.) was set at 2. Cells transduced with a SYN::GFP lentivirus were differentiated into neurons in 5 to 6 weeks of culture, fixed, and immunostained with GFP and CTIP2 antibodies. Differentiated neurons were traced using Neurolucida v. 2017 (MBF Bioscience, Williston, VT) connected to a Zeiss Axio Imager 2 microscope at a 40× oil objective. This analysis was performed only on neurons that were both GFP- and CTIP2-positive. Neurolucida Explorer v. 11 (MBF Bioscience, Williston, VT) was used to quantify the morphology of the neurons and then to obtain the sum of length of all neurites and dendrites per traced neuron. Scholl analysis was performed using Neurolucida Explorer. This analysis specified a center point within the cell body and created a grid of concentric rings around it with radii increasing in 10 μm increments. Neuronal complexity was determined by recording the number of intersections within each ring.

Quantification of synaptic spots was performed as follows.

Five- to six-week-old neurons were fixed and stained for the following markers: SYN1, postsynaptic density protein 95 (PSD-95), and dendritic marker (MAP2). A fluorescence microscope (Z1 Axio Observer Apotome Zeiss) was used to image the slides by compiling Z stack images taken from changing focus distances. Co-localized SYN1 (presynaptic) and PSD-95 (postsynaptic) were quantified using the compiled Z stack image. Only co-localized puncta that were in proximity of a MAP2-positive neurites were used in this analysis.

### Measurement of Cortical Organoid Diameter

Cortical organoids were imaged on an Evos FL Imagine System (ThermoFisher) at 4× magnification. The images were uploaded to Image J and the diameter of each organoid was determined by the program's length measurement function.

Multi-electrode array (MEA) was performed as follows.

Twelve-well MEA plates (Axion Biosystems) were coated with 100 μg/mL poly-L-ornithine (Sigma-Aldrich) and 5 μg/mL laminin (Life Technologies). Neurospheres generated from NPCs were placed in the center of the MEA wells and the electrodes were fully covered. Neurospheres were plated in NG media supplemented with 1% FBS. One week after plating, neurospheres were mixed with 1:1 of Neurobasal media and NG media, and the media was changed to the media containing 1% FBS. Two weeks after plating, neurospheres media was changed to only neurobasal and kept in this media for the rest of the recordings. Media was changed twice a week and recordings were measured one day after the media was changed. Recordings were performed once a week using a Maestro MEA System and AxIS Software (Axion Biosystems). A band-pass filter with 10 Hz and 2.5 kHz cut-off frequencies were used and a spike detector threshold was set to 5.5 times the standard deviation. For each recording, the plate was left untouched in the Maestro for 3 minutes prior to recording, and then, 3 minutes of data were recorded. Analysis was performed using Axion Biosystems Neural Metrics Tool. The criteria for detecting an activte electrode was set to 5 spikes per minute and the criterion for detecting a burst electrode was set to 5 bursts per minute.

Quantification and statistical analysis were performed as follows.

Technical replicates were used to determine standard error. N is displayed in each Figure. Standard spreadsheet software (Microsoft Excel version 15.33) was used to organize data. Error bars for Figures are standard error of the mean (S.E.M.) calculated using GraphPad Prism v6 (Graphpad Software Inc). For t-test analysis, two-tailed unpaired tests with α=0.05 was used. For multiple comparisons, significant differences were determined with ANOVA, using Tukey's multiple comparisons test. Grabbs' test with α=0.05 was performed to determine outliers, and significant values were excluded from analysis.

### Example 3

### Effects of Reverse Transcription Inhibitors for Retrotransposon L1 on Maternal Immune Activation (MIA)

### Example 3-1: Method of Preparing MIA Model Mice

The day when plug was confirmed in pregnant mice (C57BL/6NCrl) was set to day 0, and polyI:C (Sigma, P1530) dissolved in PBS or PBS as a control (CTRL) was administered intraperitoneally by syringe at 20 mg/kg body weight (once/day) for 3 days starting on day 12. The resulting male mice were used as MIA mice (MIA). For administration of the reverse transcriptase inhibitor 3TC, 3TC was dissolved in high-pressure steam-sterilized tap water at a concentration of 2 mg/ml, and mice were fed with the solution regularly from post-weaning (4 weeks old).

### Example 3-2: Behavioral Analysis of Mice Using a Three-Chamber Test to Assess Social Skills

A three-room box separated by two partitions was prepared. The two partitions have entrances and exits that allow mice to freely move in and out of the room. The rooms on either side of the central room (right and left sides) each had a cage large enough to hold a mouse. However, the cages have no entrances or exits, and the mice placed in them are not allowed to enter the cages.

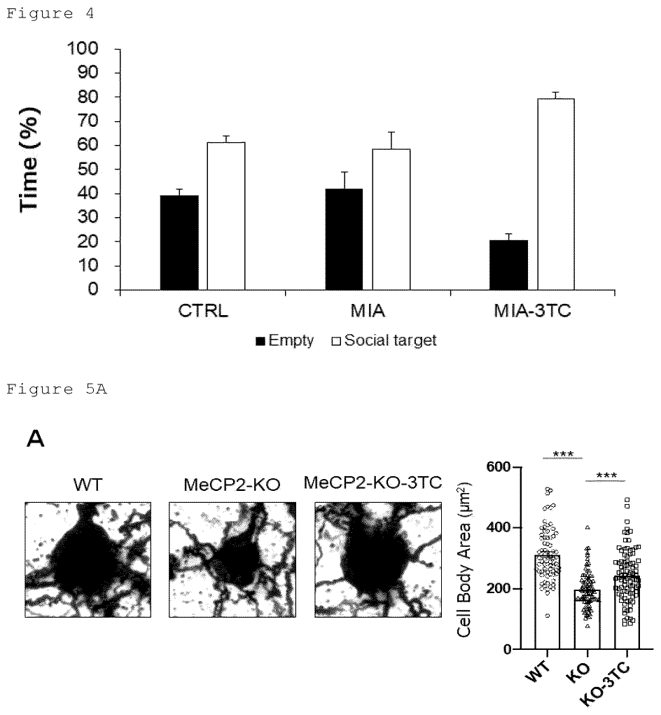

In the first session, control (CTRL) mice, MIA model mice, and 3TC-treated MIA model mice were placed in a central room as the test mice to be tested, and each was allowed to move freely for 5 minutes to acclimate to the environment, after which the test mice were returned to their original rearing cages. In the second session, male mice of the same age as the test mice (social target) were placed in the cage on the right side of the box, while the cage on the left side of the box was left empty (Empty). After 5 minutes, the test mice were released into the central room and allowed to move freely for 10 minutes, thereafter using the video analysis program to measure the approach time to each cage. Since mice are usually more interested in first-meeting mice, normal mice spend more time approaching to the cage on the right side, in which male mice of the same age as the test mice were placed. The results are shown in FIG. 4. MIA model mice showed abnormal social skills, and administration of 3TC made improved social skills.

### Example 4

### Quantification of Neuronal Cell Body Area and Spine Density Using Golgi Staining

Brains were first removed from 8-week-old WT (wild-type) mice, MeCP2-KO (KO) mice, and 3TC-treated MeCP2-KO (KO-3TC) mice, and Golgi staining was performed using FD Rapid GolgiStain™ Kit™ (FD Neuro Technologies: PK401) according to the attached protocol.

Specifically, the removed brains were rinsed in distilled water, and the tissues were infiltrated with a mixture of solution A and solution B prepared 24 hours previously and stored at room temperature in the dark for 2 weeks. The next day, the osmotic solution was replaced. After 2 weeks, they were transferred to solution C and stored for 1 week at room temperature in the dark, and the osmotic solution was also changed the next day. Mouse brains were then embedded in Cryomold No. 3 (Sansho: 83-2254) using TFM (Pharma: 303-100-1), an embedding agent for frozen tissue section preparation, and frozen at −80° C. Frozen samples were cut into 100 μm thick sections at −22° C. using a Leica CM 1900 cryostat and mounted on gelatin-coated microscope slides that had been dripped with solution C. After sectioning, the sections were allowed to dry naturally at room temperature. The sections were rinsed twice with distilled water for 4 minutes each, then transferred into a mixture of solution D:solution E:distilled water=1:1:2, and immersed for 10 minutes. The sections were rinsed twice with distilled water for 4 minutes each, and dehydrated with 50%, 75% and 95% ethanol for 4 minutes each. Then, the sections were dehydrated four times with anhydrous ethanol for 4 minutes each, permeabilized three times with xylene for 4 minutes each, and were sealed in Permount (Pharma: SP15-100-1) to prepare the specimens.

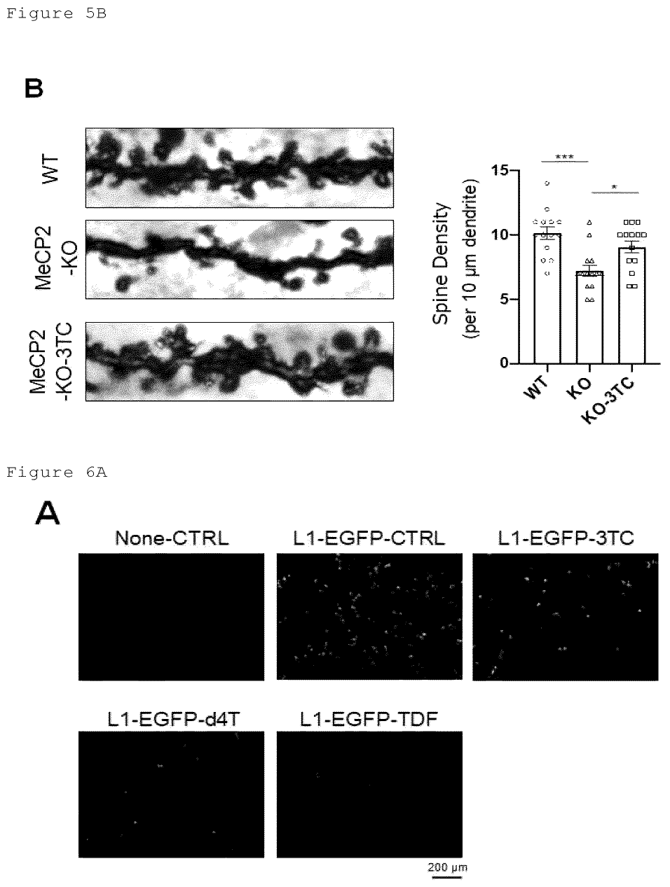

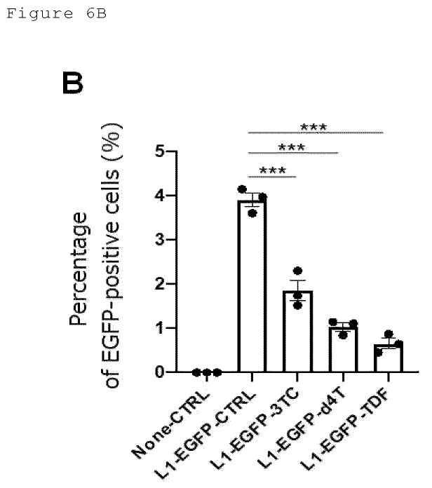

A Keyence fluorescence microscope BZ-X800 was used to acquire Golgi-stained images of mouse brain specimens. Image J was used to perform measurements, so that cell body area was quantified using a 20× objective to capture areas of the prefrontal cortex in each mouse, and spine density was quantified using a 100× objective to capture dendrites of prefrontal cortical neurons in each mouse. The results obtained are shown in FIGS. 5A and 5B, respectively. Decreases in cell body area and spine density were observed in KO mice compared to WT, but treatment with 3TC improved that phenotype.

### Example 5

Inhibitory Effects of Reverse Transcriptase with Reverse Transcriptase Inhibitors

The reverse transcriptase inhibitors 3TC, d4T and Tenofovir disoproxil Fumarate (TDF), QA-2704, Combi-Blocks) were added to cell lines derived from human fetal kidney cells (HEK293) to a final concentration of 20 μM, and the cells were introduced with a vector (L1-EGFP, Macia et al. Genome Res 27, 335-348 (2017)) in which EGFP protein was expressed in only cells with activated L1. Four days after incubating, immunostaining was performed using antibodies against GFP and Hoechest, which stains nuclei, and images were taken using a fluorescence microscope (Leica AF600) with a 10× objective. As a result, the administration of the reverse transcriptase inhibitors was found to decrease the percentage of EGFP-positive cells, confirming that the inhibition of reverse transcriptase suppressed the activity of L1.

1: A pharmaceutical composition for treating and/or preventing a developmental disorder or a mental disease, comprising:
a reverse transcription inhibitor for retrotransposon L1,
wherein the inhibitor excludes a non-nucleoside reverse transcriptase inhibitor, nevirapine.

2: The pharmaceutical composition according to claim 1, wherein the reverse transcription inhibitor for retrotransposon L1 is a reverse transcriptase inhibitor.

3: The pharmaceutical composition according to claim 2, wherein the reverse transcriptase inhibitor is a nucleoside analog reverse transcriptase inhibitor.

4: The pharmaceutical composition according to claim 3, wherein the nucleoside analogue reverse transcriptase inhibitor is lamivudine or stavudine.

5: The pharmaceutical composition according to claim 1, wherein the developmental disorder is Rett syndrome.

6: The pharmaceutical composition according to claim 1, wherein the developmental disorder is an autism spectrum disorder.

7: The pharmaceutical composition according to claim 1, wherein the mental disease is schizophrenia.

8: The pharmaceutical composition according to claim 3, wherein the nucleoside analogue reverse transcriptase inhibitor is lamivudine.

9: The pharmaceutical composition according to claim 3, wherein the nucleoside analogue reverse transcriptase inhibitor is stavudine.

10: The pharmaceutical composition according to claim 2, wherein the developmental disorder is Rett syndrome.

11: The pharmaceutical composition according to claim 2, wherein the developmental disorder is an autism spectrum disorder.

12: The pharmaceutical composition according to claim 2, wherein the mental disease is schizophrenia.

13: The pharmaceutical composition according to claim 3, wherein the developmental disorder is Rett syndrome.

14: The pharmaceutical composition according to claim 3, wherein the developmental disorder is an autism spectrum disorder.

15: The pharmaceutical composition according to claim 3, wherein the mental disease is schizophrenia.

16: The pharmaceutical composition according to claim 4, wherein the developmental disorder is Rett syndrome.

17: The pharmaceutical composition according to claim 4, wherein the developmental disorder is an autism spectrum disorder.

18: The pharmaceutical composition according to claim 4, wherein the mental disease is schizophrenia.

19: The pharmaceutical composition according to claim 8, wherein the developmental disorder is Rett syndrome or an autism spectrum disorder, and the mental disease is schizophrenia.

20: The pharmaceutical composition according to claim 9, wherein the developmental disorder is Rett syndrome or an autism spectrum disorder, and the mental disease is schizophrenia.
